(19) United States
(12) Patent Application Publication
(10) Pub. No.: US 2024/0057966 A1
(43) Pub. Date: Feb. 22, 2024

(54) ELECTRONIC DEVICE

(71) Applicant: ONKYO CORPORATION, Osaka (JP)

(72) Inventors: Makoto YOSHIDA, Osaka (JP); Yoshitada TAKESHIMA, Osaka (JP)

(73) Assignee: ONKYO CORPORATION, Osaka (JP)

(21) Appl. No.: 18/230,209

(22) Filed: Aug. 4, 2023

(30) Foreign Application Priority Data
Aug. 17, 2022 (JP) 2022-129826

(51) Int. Cl. A61B 7/04

(52) U.S. Cl. CPC A61B 7/04

(57) ABSTRACT

An electronic device comprising: a circuit; an enclosure which stores the circuit; and an earth plate which is provided at the enclosure and exposes to an outside.

### CROSS-REFERENCE TO RELATED APPLICATIONS

This application claims priority to Japanese Application No. 2022-129826, filed Aug. 17, 2022, the entire contents of which are incorporated herein by reference.

### FIELD

The present disclosure relates to an electronic device.

### BACKGROUND

In stethoscopes, there is a so-called digital stethoscope which collects sounds such as heart sounds by a sensor such as a microphone, amplifies collected sounds and makes a doctor and the like listen to amplified sounds (for example, see JP2004-242849 A). Further, there is an electrocardiograph which is for measuring an electrocardiogram. The digital stethoscope is approached to a human body so as to obtain biological sounds (auscultatory sounds) and the electrocardiograph is approached to the human body so as to obtain the electrocardiogram. Since electronic circuits are included in these electronic devices (the digital stethoscope and the electrocardiograph), there are cases where noises from a surrounding environment such as hum noises of an electric light line become a problem when the electronic circuit positions near the human body. This cause is the following.
(1) The electronic circuit and a fluctuation of a surrounding electric potential electromagnetically combine.
(2) An input circuit of a sensor is a high impedance input circuit.

In the digital stethoscope which is contacted to the human body and measures and obtains biological sounds, for example, a piezoelectric element is used. Especially, in a circuit which includes a high impedance piezoelectric element, noises are noticeable.

Like the electrocardiograph, in a device which measures an electrical potential of the human body, grounding is performed by a clip and a GND plate is provided (paragraph 0018 of JP2021-078596 A describes that a GND electrode is provided). Herein, in the digital stethoscope which does not directly measure the electrical potential of the human body, the GND plate is not provided. However, it is not prevented that the hum noises mix by approaching a high impedance input amplifier to the human body.

As described above, there is a problem the hum noises mix when an electronic device is approached to the human body.

### SUMMARY OF THE DISCLOSURE

According to one aspect of the disclosure, there is provided an electronic device comprising: a circuit; an enclosure which stores the circuit; and an earth plate which is provided at the enclosure and exposes to an outside.

### BRIEF DESCRIPTION OF THE DRAWINGS

FIG. 1 is a perspective diagram illustrating a stethoscope according to an embodiment of the present disclosure.

FIG. 2 is a perspective diagram illustrating the stethoscope according to the embodiment of the present disclosure.

FIG. 3 is an image diagram illustrating a printed substrate and circuits within an enclosure.

FIG. 4 is an image diagram illustrating the printed substrate and the circuits within the enclosure.

### DETAILED DESCRIPTION OF THE PREFERRED EMBODIMENTS

An objective of the present disclosure is to prevent mixing of hum noises.

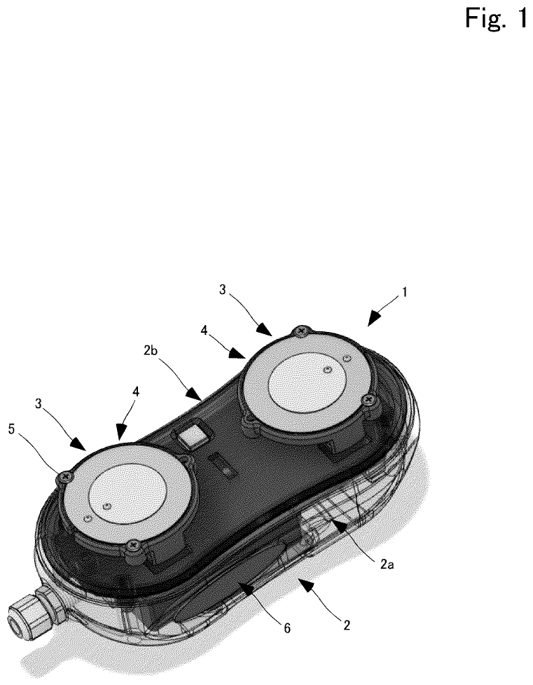

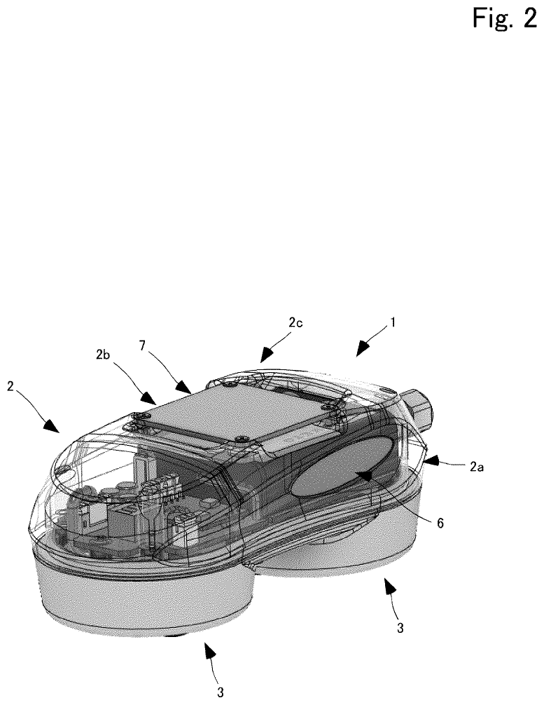

Hereinafter, an embodiment of the present disclosure is described. Each of FIG. 1 and FIG. 2 is a perspective diagram illustrating a stethoscope 1 (an electronic device) according to the embodiment of the present disclosure. It is regarded that a contact surface side that a piezoelectric element 3 is provided and contacts an auscultatory subject is a front surface and an opposite side to the front surface is a rear surface. A user of the stethoscope 1 uses the stethoscope 1 by grasping the stethoscope 1 with the hand from the rear surface side of the stethoscope 1 and contacting the front surface side the stethoscope 1 to the auscultatory subject.

The stethoscope 1 is a substantially hourglass shape. The stethoscope 1 includes an enclosure 2, the piezoelectric element 3, a holder 4 and the like. The enclosure 2 stores a printed substrate, a battery and the like therein. In the present embodiment, two piezoelectric elements 3 are provided at the front surface side of the stethoscope 1. In the present embodiment, two piezoelectric elements 3 are provided. The number of the piezoelectric elements 3 is not limited to two and may be one or more than three. The piezoelectric element 3 is a substantially flat disk shape. The piezoelectric element 3 is mounted to the holder 4.

In the present embodiment, the piezoelectric element 3 is fixed by screws 5 to the holder 4 at three points. Concretely, the screws 5 fix an outer edge (an outer circumference) of the piezoelectric element 3 to the holder 4 at three points.

As described above, the stethoscope 1 has the contact surface which contacts to a human body (a subject). The contact surface positions a place corresponding to the piezoelectric element 3 and is a shape corresponding to the piezoelectric element 3. In the present embodiment, since the piezoelectric element 3 is a substantially disk shape, the contact surface is a substantially circle shape corresponding to this. Further, since the piezoelectric elements 3 are two, the contact surfaces are also two.

The enclosure 2 has side walls 2a and 2b which respectively extend in a substantially vertical direction against the contact surfaces. Each of the side walls 2a and 2b is a part which extends in a longitudinal direction of the stethoscope 1. In other words, each of the side walls 2a and 2b is a part which extends in a direction in which the piezoelectric elements 3 are arranged. Each of the side walls 2a and 2b is recessed to the contact surfaces side, in other words, inside. Further, the enclosure 2 has an upper wall 2c which is opposed to the contact surfaces which contact to the human body.

The stethoscope 1 further includes earth plates 6. Each of the earth plates 6 is provided at the enclosure 2 and exposes from the enclosure 2 to an outside. Concretely, the earth plates 6 are respectively provided at both side walls 2a and 2b of the enclosure 2. More concretely, the earth plates 6 are respectively provided at recessed parts of the both side walls 2a and 2b.

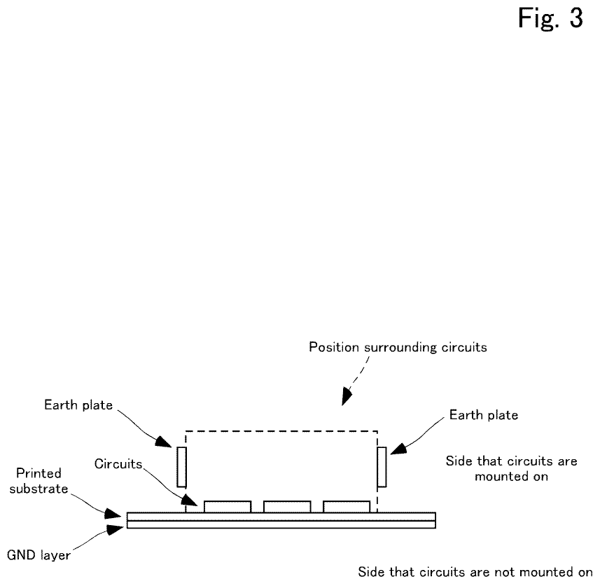

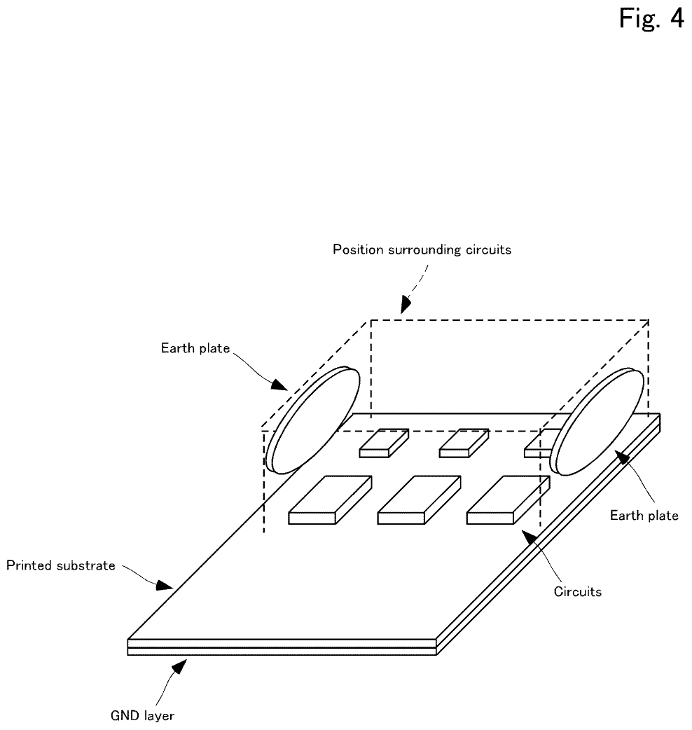

As described above, the enclosure 2 stores the printed substrate. Circuits such as an amplifier are mounted on the printed substrate. The amplifier amplifies auscultatory sounds which are collected by the piezoelectric element 3. Each of FIG. 3 and FIG. 4 is an image diagram of the printed substrate and the circuits within the enclosure 2. FIG. 3 is an image diagram which is seen from the longitudinal direction of the enclosure 2. A GND layer (a ground layer) is provided on a back side of the printed substrate. As described above, the earth plates 6 are respectively provided at the recessed parts of the both side walls 2a and 2b. These positions are in a side of the printed substrate on which the circuits are mounted and surround the circuits. Namely, the earth plates 6 are provided at positions which are in a side of the printed substrate on which the circuits are mounted and surround the circuits. Further, the earth plates 6 are provided near the circuits. High impedance circuits such as an input circuit are included in the circuits.

The earth plate 6 may be provided at the upper wall 2c. In the present embodiment, a member 7, which constitutes a belt loop into which a belt for fixing the stethoscope 1 to a human body is inserted, is provided at the upper wall 2c, however, the earth plate 6 may be provided at this position.

The “earth plate” means a plate which is for performing “earthing” and a concept which includes a “ground (GND) plate” which is for performing “grounding (GND)”.

As described above, in the present embodiment, the earth plates 6 are provided at the enclosure 2 and expose to the outside. When the user of the stethoscope 1 tries to approach the stethoscope 1 to the human body, the user grasps the enclosure 2 with the hand and approaches the stethoscope 1 to the human body. For this reason, the hand necessarily contacts to the earth plates 6. Therefore, mixing of the hum noises can be prevented.

Further, in the present embodiment, the earth plates 6 are provided at the positions which is in the side of the printed substrate on which the circuits are mounted and surround the circuits. For this reason, a loop that a ground and a signal make can be minimized and the mixing of the hum noises can be prevented more.

Further, the earth plates 6 are provided at the side walls 2a and 2b which extend in a substantially vertical direction against the contact surfaces which contact to the human body. Since the user of the stethoscope 1 uses the stethoscope 1 by grasping the side walls 2a and 2b, it is easy for the user to contact to the earth plates 6 with his/her hand.

Further, in the present embodiment, the side walls 2a and 2b of the enclosure 2 which extend in a substantially vertical direction against the contact surface which contacts to the human body are recessed to the contact surfaces side. For this reason, the user of the stethoscope 1 is easy to use the stethoscope 1 by grasping the recessed parts of the side walls 2a and 2b.

Further, in the present embodiment, the earth plates 6 are provided at the recessed parts of the side walls 2a and 2b of the enclosure 2. Since the user of the stethoscope 1 uses the stethoscope 1 by grasping the recessed parts of the side walls 2a and 2b, it is easy that the hand of the user contacts to the earth plates 6.

Further, in the present embodiment, the stethoscope 1 includes the piezoelectric element 3. Since the piezoelectric element 3 is a high impedance, a circuit to which a signal from the piezoelectric element 3 is input is also a high impedance and it is easy that the hum noises mix. However, mixing of the hum noises can be prevented by the earth plates 6.

Further, in the present embodiment, the stethoscope 1 includes the amplifier. As described above, since the piezoelectric element 3 is a high impedance, the amplifier which receives a signal from the piezoelectric element 3 and amplifies the signal is also a high impedance and it is easy that the hum noises mix. However, mixing of the hum noises can be prevented by the earth plate 6.

Further, the earth plate 6 may be provided at the upper wall 2c of the enclosure 2 which is opposed to the contact surface which contacts to the human body. Since the user of the stethoscope 1 contacts the contact surface to the subject by grasping the enclosure 2, it is easy that the hand of the user contacts to the earth plate 6 which is provided at the upper wall 2c of the enclosure 2 opposed to the contact surface.

The embodiment of the present disclosure is described above, but the mode to which the present disclosure is applicable is not limited to the above embodiment and can be suitably varied without departing from the scope of the present disclosure.

The present disclosure can be suitably employed in an electronic device.

What is claimed is:

1. An electronic device comprising:
a circuit;
an enclosure which stores the circuit; and
an earth plate which is provided at the enclosure and exposes to an outside.

2. The electronic device according to claim 1 further comprising
a substrate on which the circuit is mounted,
wherein the enclosure stores the substrate, and
the earth plate is provided at a position which is in a side of the substrate on which the circuit is mounted and surrounds the circuit.

3. The electronic device according to claim 1,
wherein the enclosure has a side wall which extends in a substantially vertical direction against a contact surface which contacts to a subject, and
the earth plate is provided at the side wall.

4. The electronic device according to claim 1,
wherein the enclosure has a side wall which extends in a substantially vertical direction against a contact surface which contacts to a subject, and
the side wall is recessed to a contact surface side.

5. The electronic device according to claim 4,
wherein the earth plate is provided at a recessed part of the side wall.

6. The electronic device according to claim 1,
wherein the enclosure has an upper wall which is opposed to a contact surface which contacts to a subject, and
the earth plate is provided at the upper wall.

7. The electronic device according to claim 1 further comprising:
a piezoelectric element,
wherein a signal from the piezoelectric element is input to the circuit.

8. The electronic device according to claim 1,
wherein the circuit includes an amplifier which amplifies a signal from the piezoelectric element.

9. The electronic device according to claim 1,
wherein the electronic device is a stethoscope.

10. The electronic device according to claim 2,
wherein the substrate includes a GND layer.
